(19) United States
(12) Patent Application Publication
(10) Pub. No.: US 2017/0347919 A1
(43) Pub. Date: Dec. 7, 2017

(54) Micro deviation detection device

(71) Applicants: Jimmy Dale Bollman, Oklahoma City, OK (US); Victoria Marisia Rossiter, Edmond, OK (US); Scott Raymond Kincannon, Edmond, OK (US); Alex William Greenfeather, Edmond, OK (US)

(72) Inventors: Jimmy Dale Bollman, Oklahoma City, OK (US); Victoria Marisia Rossiter, Edmond, OK (US); Scott Raymond Kincannon, Edmond, OK (US); Alex William Greenfeather, Edmond, OK (US)

(21) Appl. No.: 14/999,603

(22) Filed: Jun. 1, 2016

(51) Int. Cl. A61B 5/107; A61B 5/11; A61B 5/00; H04W 4/00; G06F 19/00

(52) U.S. Cl. CPC A61B 5/1071; H04W 4/008; G06F 19/345; A61B 5/7282; A61B 5/742; A61B 5/002; A61B 5/4836; A61B 5/407; A61B 5/11; A61B 5/1075; A61B 2562/0219

(57) ABSTRACT

A early detection device to diagnose scoliosis in adolescents without exposure to harmful x-rays provides a portable, accurate and inexpensive hand-held diagnostic tool to determine deviations from the Cobb Angle in the spine, the detection device connecting to a receiver unit to receive data obtained from the detection device completing the diagnostic testing for visual review and diagnosis on the receiver unit. It is also provided in alternative embodiment to test the integrity of structural components in various industries, including surface irregularities and defects and cracks in beams and wall, pipes or beams.

### CROSS REFERENCE TO RELATED APPLICATIONS

None.

### I. BACKGROUND OF THE INVENTION

### 1. Field of Invention

A early detection device to diagnose scoliosis in adolescents without exposure to harmful x-rays provides a portable, accurate and inexpensive hand-held diagnostic tool to determine deviations from the Cobb Angle in the spine, the detection device connecting to a receiver unit to receive data obtained from the detection device completing the diagnostic testing for visual review and diagnosis on the receiver unit. It is also provided in alternative embodiment to test the integrity of structural components in various industries, including surface irregularities and defects and cracks in beams and wall, pipes or beams.

### 2. Description of Prior Art

A preliminary review of prior art patents was conducted by the applicant which reveal prior art patents in a similar field or having similar use. However, the prior art inventions do not disclose the same or similar elements as the present non-invasive diagnostic detection device, nor do they present the material components in a manner contemplated or anticipated in the prior art.

Several manual gauges are demonstrated in the prior art which visually determine a deviation of normal spinal curvature in devices that relate to scoliosis diagnosis. These devices include U.S. Pat. No. 4,033,329 to Gregory, which is a machine that one stands upon, but is used to determine C1 subluxation. U.S. Pat. No. 2,930,133 to Thompson is a device that is placed behind a patient with the arm placed upon the back starting with the lowest vertebrae and working up measuring curvature using a protractor. U.S. Pat. No. 5,181,525 to Bunnel is a device operated by running the device along the back of a patient while they are bent over with an internal plumb bob inside showing the degree of shift from center. In U.S. Pat. No. 5,156,162 to Gerhardt, a caliper is used to measure deviation from the left and right rib hump using an inclinometer to measure the angle on the spine at any given height. A spinal anthropometer or lordosimeter is described which give a 3D configuration of the spine using point encodement from spinal landmarks and then uses an algorithm to provide analysis.

A computer graph generated by a device run along the back provides a 3D graph of the spine in U.S. Pat. No. 7,883,477 to Ichikawa. A hand-held device worn on the index finger provides a position sensor knowing the predetermined distance from the tip of the index finger to track a course along the spine, tracking its course to assess curvature of the spine in U.S. Pat. No. 6,524,260 to Shechtman. An X-axis and Y-axis providing the device using a computer mouse using a roller ball and light emitter system to track motion along the spine and relate it to the X and Y axis is disclosed in U.S. Patent Application Pub. No. 2006/0015042 to Linial. Lastly, in U.S. Patent Application Pub. No. 2006/0021240 to Horgan, an inclinometer placed on the upper region of the patient transfers data to a display, the inclinometer attached to a bed frame upon which the patient lays, transfers data to computer which provides an image in green versus red contrast to determine deviation of the spine.

### II. SUMMARY OF THE INVENTION

Scoliosis is a common problem among adolescents and often goes without proper treatment due to x-rays being the primary method of diagnosis in children within this age grouping. Radiation from x-rays can be damaging to a child's developing body. This diagnosis is important especially prior to puberty where undiagnosed scoliosis can become permanent and less successfully remediated. Early detection is key with scoliosis and although there are other methods for the detection of scoliosis, the only reliable methods of measuring the degree of curvature of the spine is with x-rays or MRI scans. Since 1 in 33 people have some degree of scoliosis, it is important to develop a method of diagnosis that can be used to general checkups without special training, expensive equipment or exposure to harmful radiation.

Current scoliometers used in screenings are largely unreliable. Many of the methods used, such as the scoliometers identified in the prior art, commonly give false readings and therefore produce unreliable medical data. They are not considered legitimate data to support an official medical diagnosis. To acquire a reliable accurate measurement, a patient must receive x-rays to image their spine and then have a doctor make hand measurements to determine the Cobb Angle at any point.

Cobb angle or Cobb's angle is know in the field of medicine and is used to quantify the magnitude of spinal deformities, especially in the case of scoliosis. The angle may be plotted manually or digitally and scoliosis is defined as a lateral spinal curvature with a Cobb angle of 10 degrees or more. The Cobb angle was first described in 1948 by Dr. John R. Cobb. In an article critical of school-based screening programs in 2002, Dr. K Allen Greiner because of a lack of follow-up treatment he deemed necessary for these programs to have any value. He recommended that physicians follow up on the school diagnosed children with an MRI or radiographs of the full spine before eight years of age, for those having rapid curve progression of more than 1 degree per month, an unusual curve pattern such as left thoracic curve, neurologic deficit or pain. Early treatment of the adolescent idiopathic scoliosis is necessary to prevent the progression of the curve magnitude and should be followed up.

Additionally, industrial applications are presented in various embodiments of the detection devices for application to surface irregularities in outer surfaces of machines and building structural defects in panels, pipes, beams, and other support structures. The detection device can perform integrity diagnostics without destructive testing, being able to detect micro-defects which cannot be seen by the naked eye or visual inspection. The remote capabilities also provide the user with an ability to send real time information which can be enhanced and calibrated for further evaluation and reading by a remote diagnostic location.

The present micro deviation detection device provides a reliable pre-screening tool without unnecessary radiation for those adolescents in order to make proper referral to medical professionals during that period of time that minimization of the curve magnitude, if present, can be treated and halted prior to adulthood, where sever complications and decay of the spinal integrity can no longer be successfully treated. It also supplies a reliable diagnostic tool for the non-destructive testing of building and structural components in industrial applications.

### III. DESCRIPTION OF THE DRAWINGS

The following drawings are submitted with this utility patent application.

FIG. 1 is a first embodiment of the flat profile deviation detection device.

FIG. 2 is a sectional view of the flat profile deviation detection device along sectional lines 2/2 of FIG. 1.

FIG. 3 is an upper perspective view of the flat profile deviation detection device.

FIG. 4 is an inner view of the flat profile deviation detection device with the protective cover panel removed.

FIG. 5 is a perspective view of a second embodiment of the L-shaped deviation detection device.

FIG. 6 is a side view of the L-shaped detection device.

FIG. 7 is a sectional view of the L-shaped deviation defection device along sectional lines 7/7 of FIG. 6.

FIG. 8 is a view of the L-shaped deviation detection device with the protective cover panel removed.

FIG. 9 is a sectional view of a third embodiment of the U-shaped deviation detection device.

FIG. 10 is a lower perspective view of the U-shaped deviation detection device.

FIG. 11 is a view of the U-shaped deviation detection device with the protective cover panel removed.

FIG. 12 is a circuit diagram of the electrical components used in all three embodiments of the deviation detection device.

### IV. DESCRIPTION OF THE PREFERRED EMBODIMENT

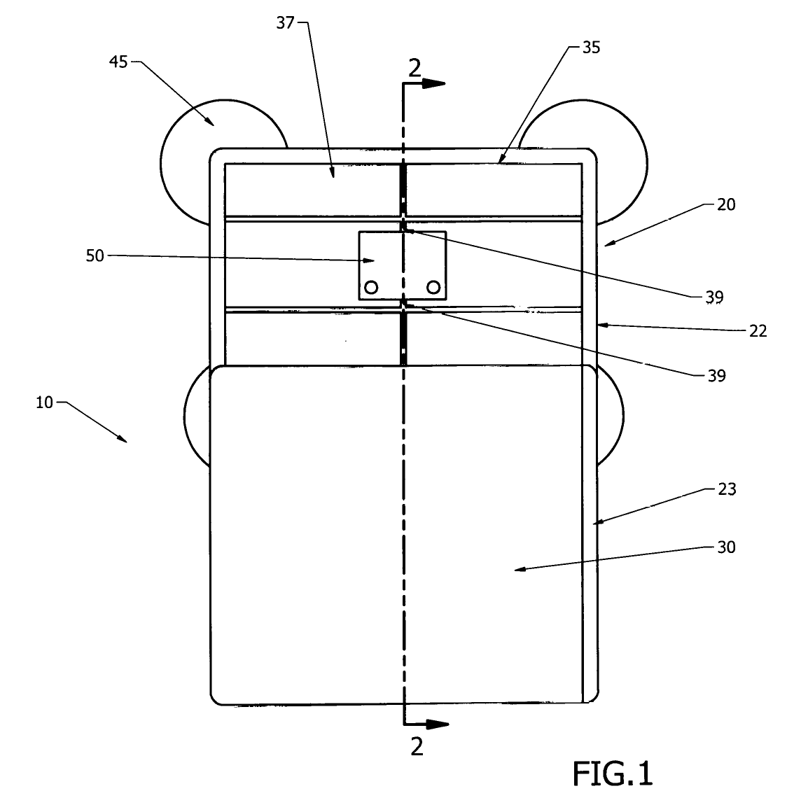

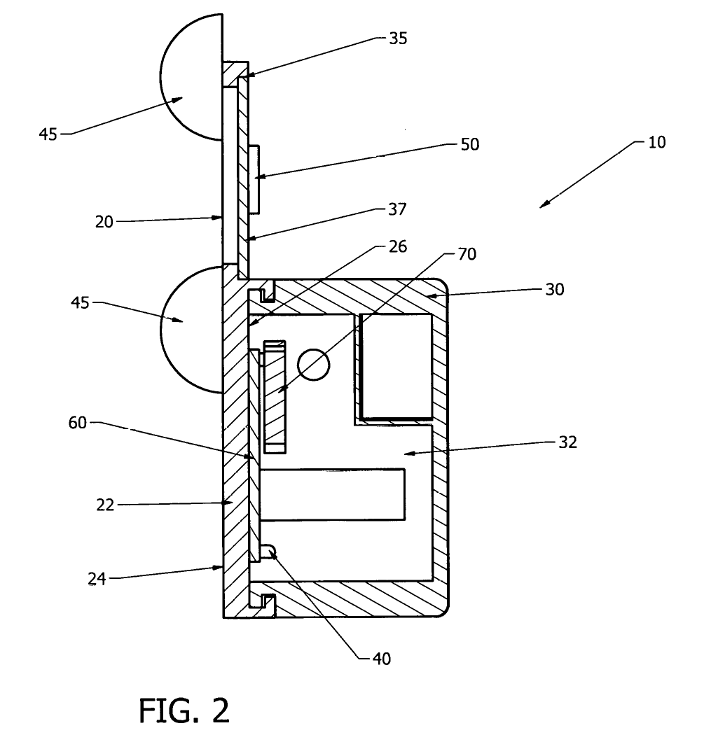

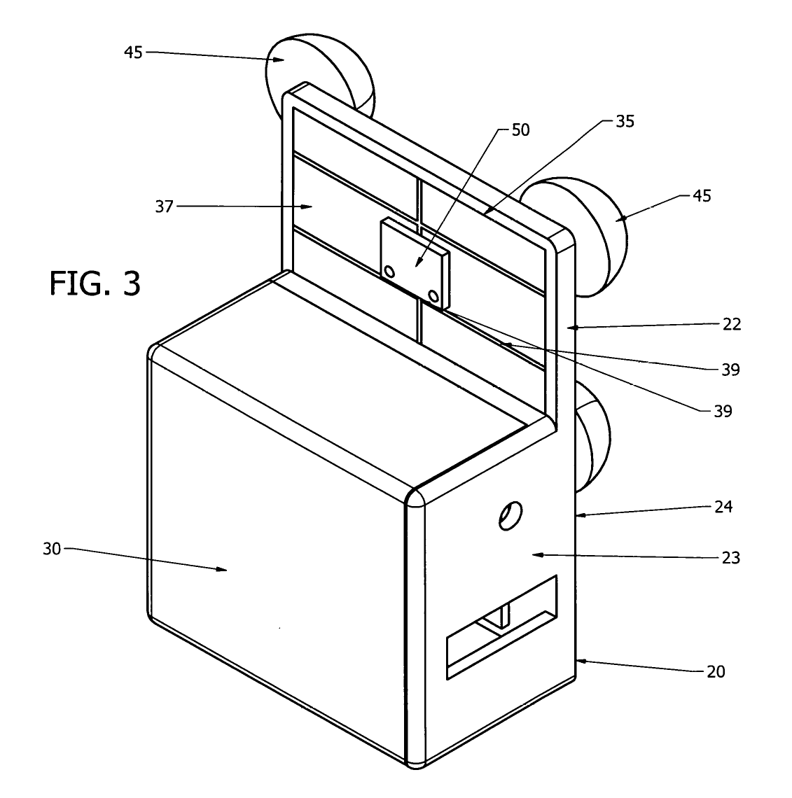

A deviation detection scan device 10 providing for the measurement of surface linear and planar deviations in certain objects, shown in FIGS. 1-12 of the drawings, the device used for the determination of micro-measured irregularities in the surface of the object using a shaped platform 20, 120, 220, a gyroscope supplied by an Inertial Measurement Unit (IMU) 50, 150, 250, a microprocessor 60/160/260, a Bluetooth module 70/170/270, for the conveyance of data from the IMU to the microprocessor and to an external computer or other visual graphic display, support pegs 40/140/240, to prevent movement of the internal components, vibration dampening sorbothane hemispheres 45/145/245, and in some embodiments, a rigid clear plastic window 37 for accurate measurements along a designated pathway on the object being measured, and graphic crosshairs 39 located upon the plastic window 37 for guidance along the designated measurement pathway.

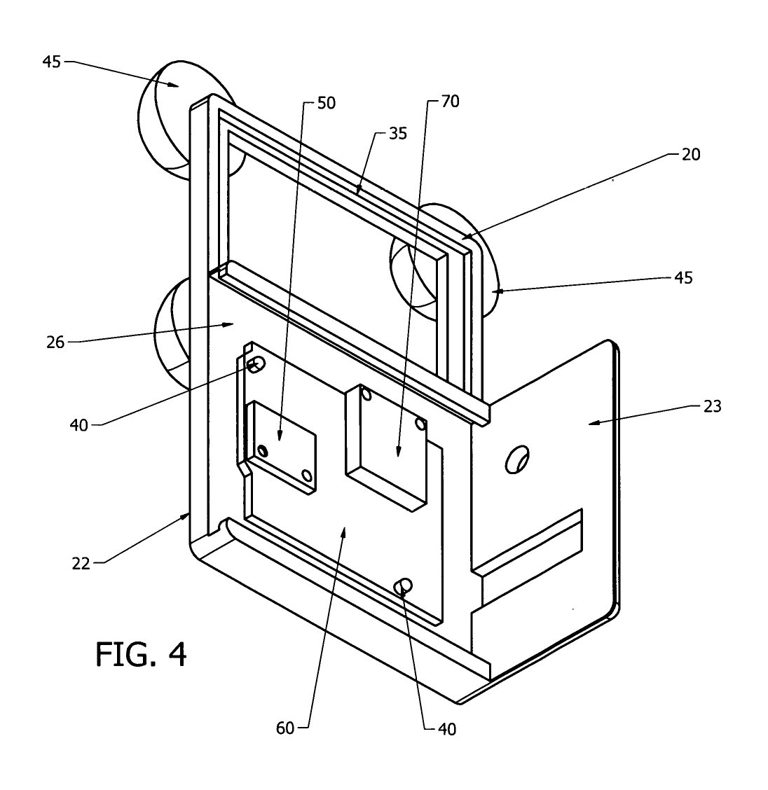

A first embodiment deviation detection device 10 is presented for the measurement of a planar surface, shown in FIGS. 1-4. In this first embodiment, the platform 20, shown in FIGS. 1-4 provides a flat base member 22 extending a side projection 23 and a removable protective cover 30, absent in FIG. 4 to reveal an inner cavity 32 and inner surface 26 of the platform base member 22. Within the flat platform base member 22 is an upper window section 35 receiving the rigid plastic window 37 including the graphic crosshairs 39. Optionally included on the plastic window 37 is at least one first IMU 50, which is preferably an electronic gyroscope. This IMU/electronic gyroscope 50 measures all the angles and deviations along an X, Y and Z axis (known as pitch, yaw, and roll) to the nearest 100^{th} of a degree as the device 10 runs along the designated pathway.

An outer surface 24 of the flat base member 22 extends a plurality of sorbothane hemispheres 45 to provide a vibration damper to the device 10 as the device 10 is run along a planar surface, minimizing human error resulting from shaking or unsteady hands. Suspended within the inner cavity 32 of the platform base member 22 by a plurality of fixed support pegs 40 is the microprocessor 60, which is an Arduino or similar microprocessor unit, a second IMU 50 and the Bluetooth module 70. The microprocessor 60 as defined herein, is preferably Arduino Uno, which has the potential to perform the functions as previously stated herein. The term Arduino, as defined herein, is an open-source prototyping platform based upon easy-to-use hardware and software, which enable circuitry to read inputs and turn it into an output. These are programmable by the user by sending a set of instructions to the microprocessor 60 using Arduino language and programming. Use of other microprocessors 60 providing the same or similar function can serve as alternative microprocessors and can be used interchangeably, which is why the component is referenced by its generic name instead of the more specific term Arduino. Thus, for purposes of this application, microprocessor 60, Arduino and Arduino-compatible are interchangeable by preference to avoid limitations of the subject matter and scope of this component. The microprocessor 60 is the main processing unit communicating all data from the IMU/gyroscope 50 to either a computer, not shown, or the Bluetooth module 70. The Bluetooth module 70 is used to relay information and data from the microprocessor to an external device, not shown, which may be a remote computer or phone. Compliant and appropriate software will convert the information and data into a meaningful chart, graph or other visual readout to determine if an abnormal findings are obtained during the operation.

The purpose of the first embodiment of the deviation detection device 10 with the flat base member 20 is for use to diagnose scoliosis in children and adolescent without exposure to harmful radiation used in other forms of diagnosis including disruptive diagnostic testing including X-ray, MRI and CT scans. Secondary application of this embodiment include non-destructive testing of manufactured products to detect fractures or dents in surfaces, including aircraft and automotive surfaces, deflections in pipes and beams, cracks in bridge supports, walls and panels, and other surface irregularities.

The present flat base deviation detection device provides a reliable pre-screening tool without unnecessary radiation for those adolescents in order to make proper referral to medical professionals during that period of time that minimization of the curve magnitude, if present, can be treated and halted prior to adulthood, where sever complications and decay of the spinal integrity can no longer be successfully treated. This device is moved along the back of the subject along the spinal points indicating the rear projection of each vertebrae. The sorbothane hemispheres 45 are in contact with the subject, and the device will be run as many times as necessary to determine if there are any deviations of the Cobb Angle, previously described in the specification, of one or more vertebrae and to what degree, this detection performed by micro-deviation shifts detected by the IMU/gyroscope 50. It will disclose a shift in the vertebrae laterally, front to back, or rotationally, and should provide an instant readout. In the event significant vertebral deviation is determine, that subject may be referred for more intense diagnosis and treatment. In the event that no deviation is determined, the subject may be cleared from further treatment. For mass pre-screening for scoliosis in a public setting, this device 10 is quick, harmless and quite efficient, having the capacity to indicate deviation to the nearest 1/100 of a degree.

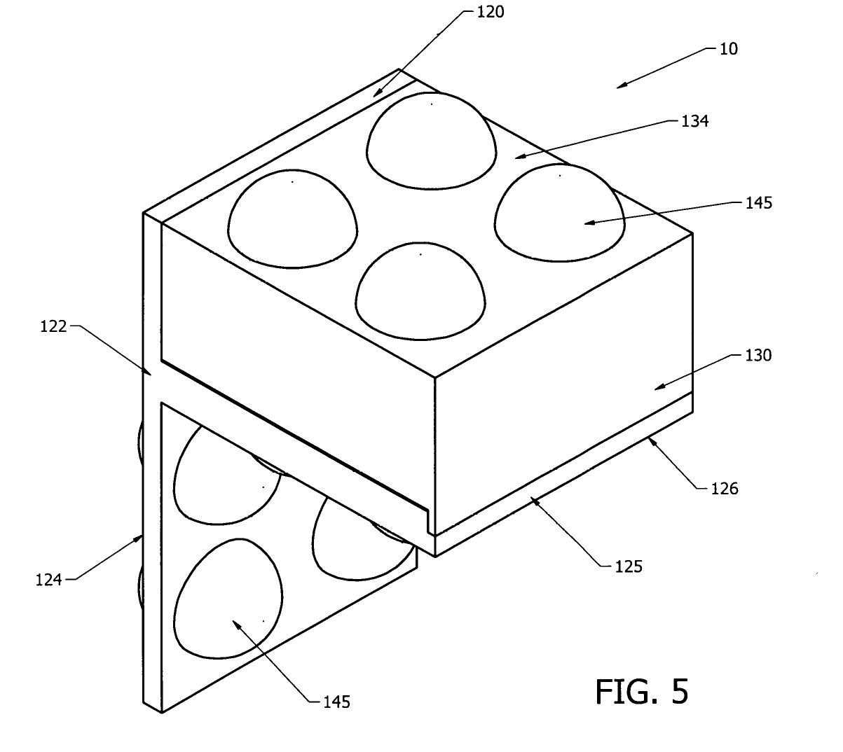

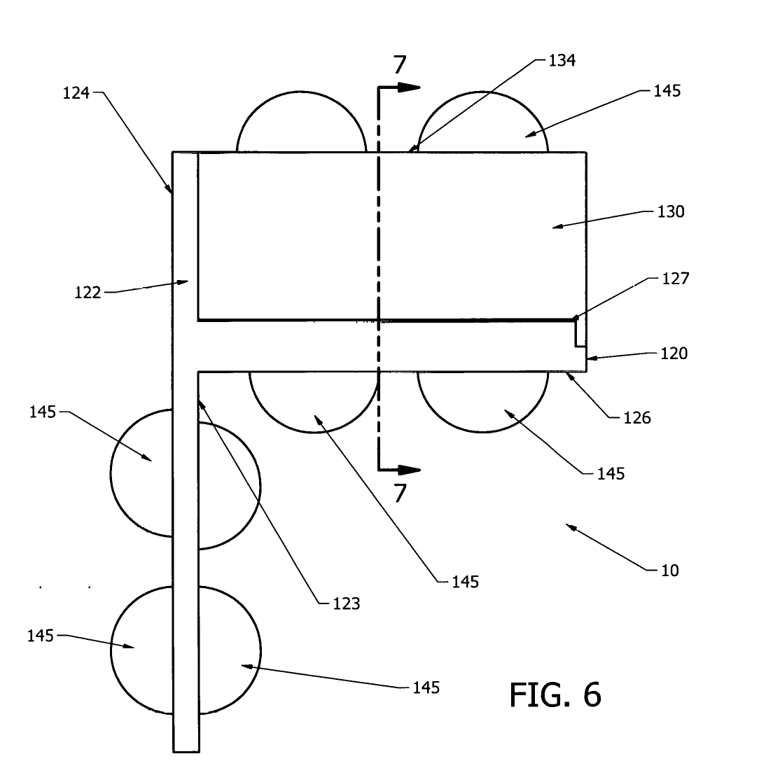

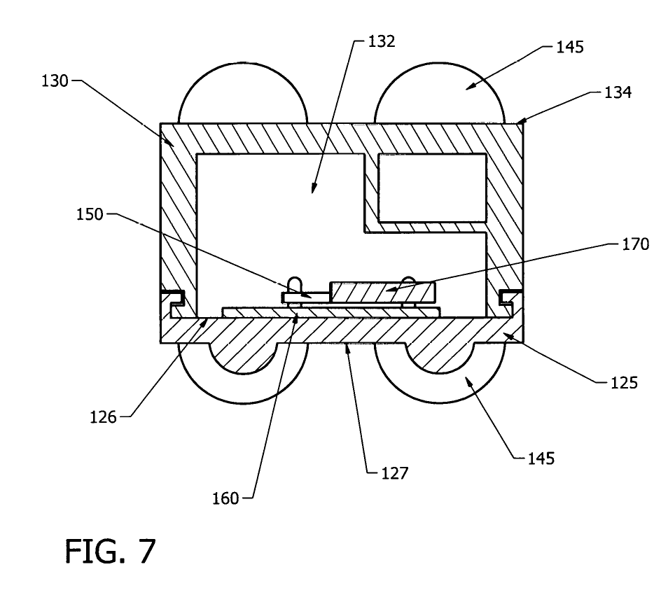

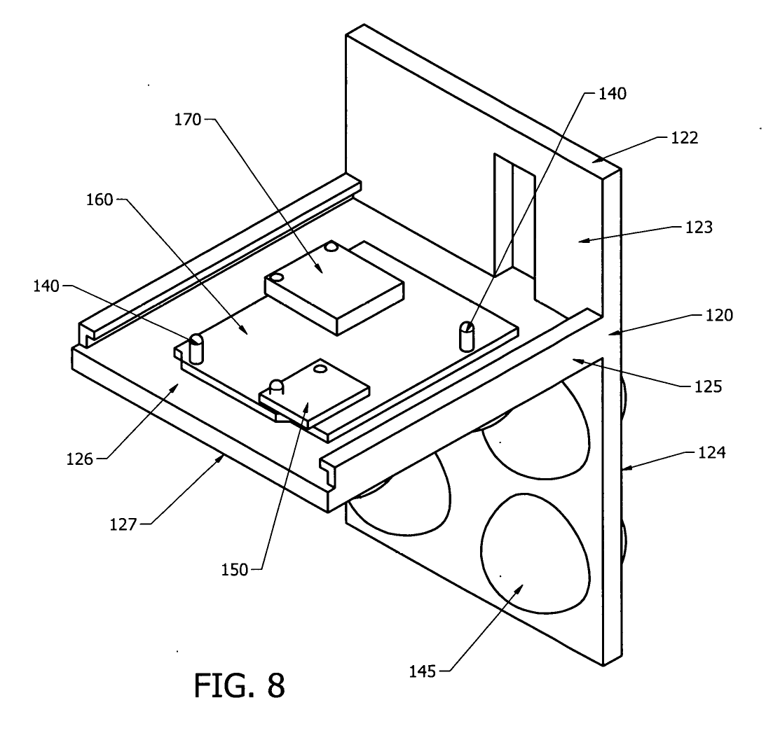

A second embodiment of the deviation detection device 10, shown in FIGS. 5-8 of the drawings, is used for the detection of irregularities in inside and outside corners. The L-shaped platform 120 defines a rear panel 122 having an inner surface 123 and an outer surface 124 and an integrated perpendicular extension panel 125 extending from the inner surface 123 defining an upper surface 126 and a lower surface 127, and a removable protective cover 130 defining an outer surface 134. Within the upper surface 126 and inside the removable protective cover 130 is an inner cavity 132, FIGS. 7-8. Upon the outer surface 124 of the rear panel 122, a portion of the inner surface 123 of the rear panel 122 not contained within the inner cavity 132, the lower surface 127 of the extension panel 125 and an outer surface 134 of the protective cover 130, as shown in FIG. 6, are a plurality of sorbothane hemispheres 145 serving the same purpose as was disclosed in the first embodiment of the deviation detections device—to minimize human error during the diagnostic testing using this second embodiment. When running diagnostics on an inside corner, the L-shaped platform is run utilizing the sorbothane hemispheres 145 located on the outer surface 134 of the protective cover 130 and the outer surface 124 of the rear panel 122. When running diagnostics on an outside corner, the sorbothane hemispheres 145 on the lower surface 127 of the extension panel 125 and the inner surface 123 of the rear panel 122 are utilized. This device 10 will determine the integrity and linear formation of each corner, which is useful in wall formation, structural component alignment, and whether a corner is joined in a straight line. It is also useful in cabinetry, furniture construction, and other woodworking matters.

As shown in FIGS. 7 and 8, the upper surface 126 of the extension panel 125 within the inner cavity 132 contains the IMU/gyroscope 150, the microprocessor/Arduino 160, the Bluetooth module 170 supported by a plurality of support pegs 140 for the stable support of the electronic components within the inner cavity 132. The primary application for this second embodiment of the L-shaped deviation detection device 10 is for the non-destructive testing of manufactured products to detect fractures or dents in corners in beams and walls, deflections in outer surfaces of pipes and beams, structural cracks in bridge supports, walls and panels, and other angular irregularities. It may also be used on cylindrical objects to diagnose uniform outer diameters and surfaces. The IMU 150, microprocessor 160 and Bluetooth module 170 function in the same manner as described in the first embodiment deviation detection device.

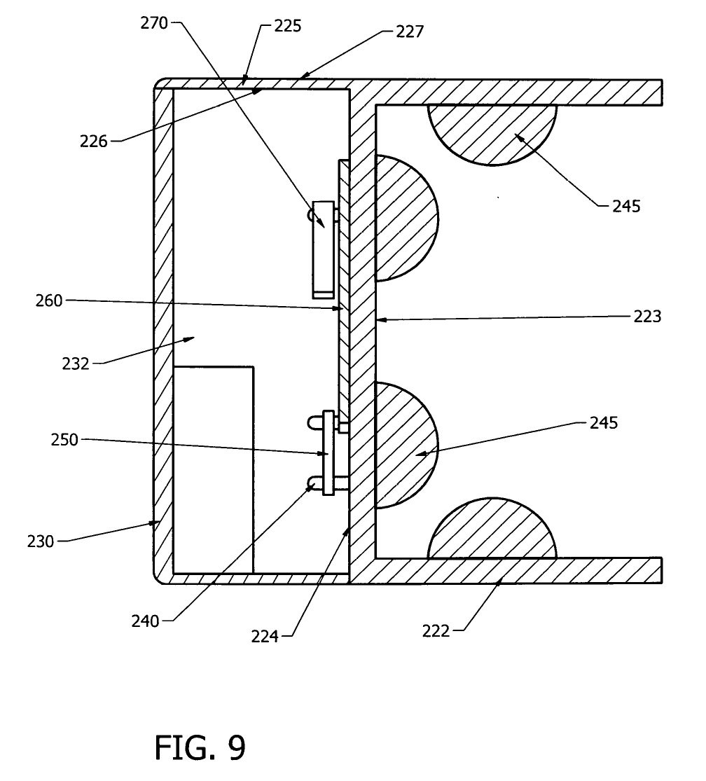

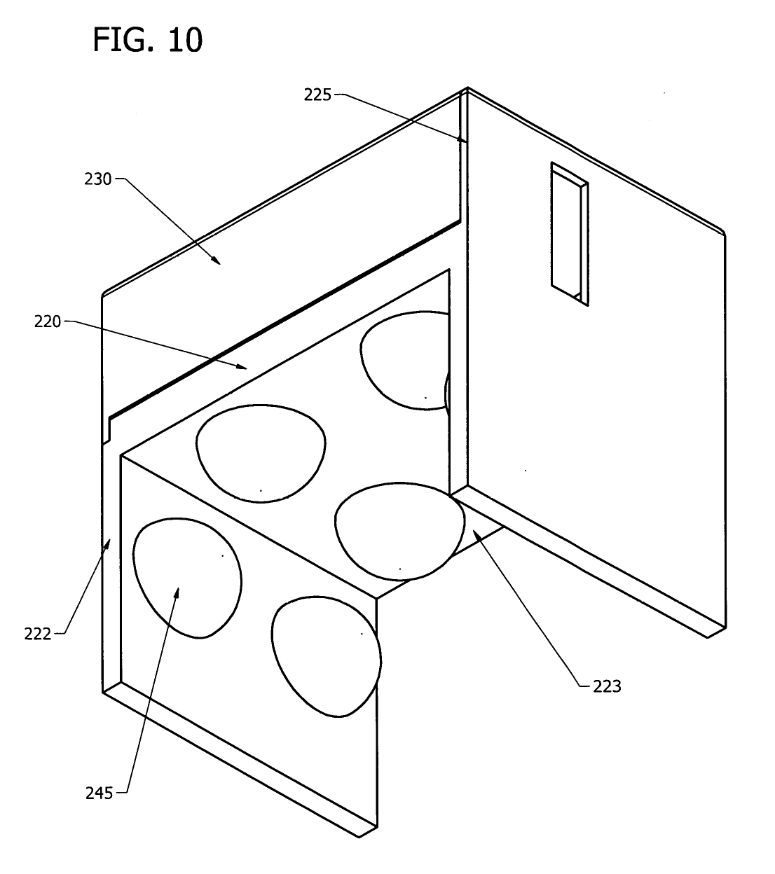

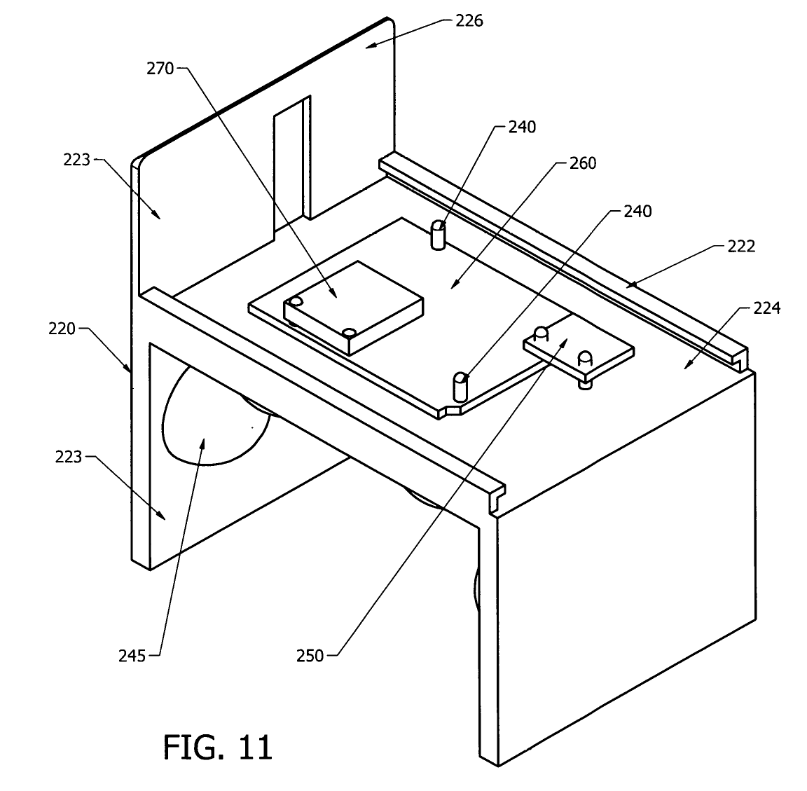

In yet a third embodiment of the deviation detection device 10 used for diagnosing small diameter outer surface irregularities in pipes and beams, as shown in FIGS. 9-11 of the drawings, a U-shaped platform 220 defines a U-shaped platform base 222 defining a U-shaped inner surface 223 and a flat cavity surface 224, a backplate 225 from the flat cavity surface 224 having an inner surface 226 and an outer surface 227, and a removable protective cover 230. An inner cavity 232 is defined by the inner surface 226 of the backplate 225, the flat cavity surface 224 from the U-shaped platform 220 and the removable protective cover 230, the inner cavity 232 exposed in FIGS. 9 and 11. Along the U-shaped inner surface 223 of the U-shaped platform base 222 is a plurality of sorbothane hemispheres 245, as demonstrated in FIGS. 9-10. When run along the outer surface of a small diameter pipe or beam, the sorbothane hemispheres 245, once again, attempt to stabilize movement and avoid human error during the diagnostic procedure of the object.

As shown in FIGS. 7 and 8, the flat cavity surface of the U-shaped platform 220 within the inner cavity 232 contains the IMU/gyroscope 250, the microprocessor/Arduino 260, the Bluetooth module 270 supported by a plurality of support pegs 240 for the stable support of the electronic components within the inner cavity 232. The IMU 250, microprocessor 260 and Bluetooth module 270 function in the same manner as described in the first embodiment deviation detection device. The primary application for this third embodiment as the U-shaped deviation detection device 10 is for the non-destructive testing of manufactured products to detect fractures or dents in outer surfaces in small diameter beams and pipes, deflections in outer surfaces of pipes and beams, structural cracks in pipes and beams and angular irregularities small dimensional objects which are capable of being fully or partially fit within the U-shaped inner surface. It may also be used on cylindrical objects to diagnose uniform outer diameters and surfaces. It is also useful to determine proper fitting and angle of joints, dimensional lumber straightness and uniformity, and bulging or pressure deviation is pipes, tubing and ducts.

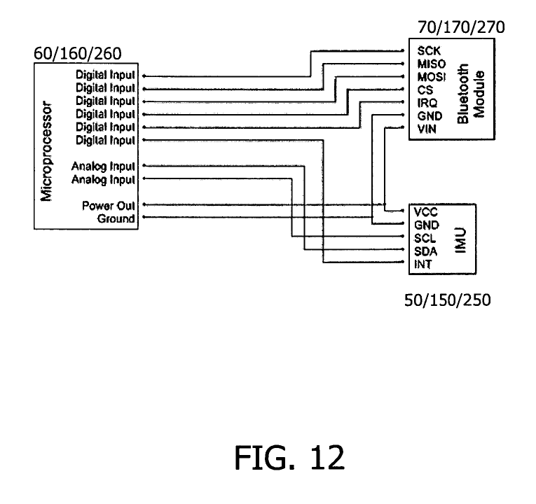

The circuit diagram shown in FIG. 12 of the drawings, demonstrates the circuit diagram for the microprocessor/Arduino 60/160/260, the IMU/gyroscope 50/150/250 and the Bluetooth module 70/170/270. Also included, but not shown, would be a power supply, either local or remote, providing a low voltage power supply suitable for the operation of the electronic circuitry shown in FIG. 12. This may be supplied by a rechargeable, low-voltage battery or external power supply means, connecting through a USB cable or other low voltage connector, not shown. Low voltage is defined herein as a power supply of less than 27 volts.

While the deviation detection devices 10, in one or more embodiments, have been particularly shown and described with reference to a preferred embodiment thereof, it will be understood by those skilled in the art that changes in form and detail may be made therein without departing from the spirit and scope of the invention.

What is claimed is:

1. A deviation detection scan device providing for the measurement of deviations in linear and planar surfaces of flat or shaped objects, resulting in a visual diagnosis of micro-measured irregularities in said surfaces of said objects, comprising:
a shaped platform defining at least one surface extending a plurality of hemispheric dampeners attached to said surface of said platform;
at least one Inertial Measurement Unit (IMU) supplied as a gyroscope;
a programmable microprocessor, and;
a Bluetooth module to convey data from said IMU to said microprocessor and to an external visual graphic display device.

2. The deviation detection scan device as disclosed in claim 1, wherein said microprocessor is an Arduino Uno, which is an open-source prototyping platform based upon easy-to-use hardware and software, which enable circuitry to read inputs and turn it into an output and are programmable by a user by sending a set of instructions to said microprocessor using Arduino language and programming.

3. The deviation detection scan device as disclosed in claim 1, further comprising:
said platform defines a flat base member having an inner surface, an outer surface and extending a side projection, a removable protective cover containing an inner cavity and an upper window section;
a rigid clear plastic window providing a plurality of graphic crosshairs, said clear plastic window set within said upper window section for accurate measurements along a designated pathway on a planar object being measured;
at least one first IMU providing an electronic gyroscope set upon said clear plastic window measuring any and all the angles and deviations along an X, Y and Z axis to the nearest 100^{th} of a degree as said device is runs along a predetermined pathway;
said plurality of hemispheres are sorbothane, providing a vibration damper to said device as said device is run along said planar object, minimizing human error resulting from shaking or unsteady hands and are mounted upon said outer surface of said flat base member;
a plurality of fixed support pegs supporting said microprocessor, said second IMU and said Bluetooth module within said inner cavity, said second IMU providing additional measuring of any and all the angles and deviations along an X, Y and Z axis to the nearest 100^{th} of a degree as said device is runs along a predetermined pathway;
said microprocessor further defining an open-source prototyping platform based upon easy-to-use hardware and software, which enable circuitry to read inputs and turn it into an output, programmable by a user by sending a set of instructions to the microprocessor using compatible language and programming, said microprocessor comprising the main processing means of communicating of all data from said first and second IMU to said Bluetooth module, said Bluetooth module used to relay information and data from the said first and second IMU to said microprocessor and to said external computer or other visual graphic display, with said Bluetooth programmed with compliant and appropriate software convert information and data into a visual chart, graph or other visual readout to determine if an abnormal findings are obtained during operation of said device, wherein said device is used to diagnose scoliosis in children and adolescent without exposure to harmful radiation used in other forms of diagnosis including disruptive diagnostic testing including X-ray, MRI and CT scans and for non-destructive testing of manufactured products to detect fractures or dents in surfaces, including aircraft and automotive surfaces, deflections in pipes and beams, cracks in bridge supports, walls and panels, and other surface irregularities.

4. The deviation detection scan device as disclosed in claim 1, used for detection of irregularities upon inside and outside corners of said object, said device further comprising:
said platform is L-shaped defining a rear panel having an inner surface and an outer surface, an integrated perpendicular extension panel extending from said inner surface, said extension panel defining an upper surface and a lower surface, and a removable protective cover defining an outer surface, said upper surface and said removable protective cover defining an inner cavity;
said plurality of hemisphere dampers are sorbothane to minimize human error during the diagnostic testing provided upon said outer surface of said rear panel, a portion of said inner surface of said rear panel outside said inner cavity, said lower surface of said extension panel and an outer surface of said protective cover;
a plurality of fixed support pegs supporting said microprocessor, said at least one IMU and said Bluetooth module within said inner cavity an upon said upper surface of said extension panel, providing additional measuring of any and all the angles and deviations along an X, Y and Z axis to the nearest 100^{th} of a degree as said device is runs along a predetermined pathway;
said microprocessor further defining an open-source prototyping platform based upon easy-to-use hardware and software, which enable circuitry to read inputs and turn it into an output, programmable by a user by sending a set of instructions to the microprocessor using compatible language and programming, said microprocessor comprising the main processing means of communicating of all data from said at least one IMU to said Bluetooth module, said Bluetooth module used to relay information and data from the said at least one IMU to said microprocessor and to said external computer or other visual graphic display, with said Bluetooth programmed with compliant and appropriate software convert information and data into a visual chart, graph or other visual readout to determine if an abnormal findings are obtained during operation of said device, wherein said L-shaped platform is used for diagnostic testing of a linear inside corner utilizing the sorbothane hemisphere dampers located on said outer surface of said protective cover and said outer surface of said rear panel and, when diagnostic testing of a linear outside corner, said hemisphere dampers on said lower surface of said extension panel and said inner surface of said rear panel are utilized, said device used to determine the integrity and linear formation of each said linear inside and outside corners, said L-shaped platform useful in diagnostic testing of wall formations, structural component alignment, and whether a corner is joined in a straight line or used on cylindrical objects to diagnose uniform outer diameters and surfaces.

5. The deviation detection scan device as disclosed in claim 1, used for detection and diagnosis of irregularities in small diameter pipes and beams said shaped objects, said device further comprising:
said platform is U-shaped and defines a U-shaped platform base with a U-shaped inner surface and a flat cavity surface, a backplate extending from said flat cavity surface, said backplate defining an inner surface and an outer surface, and a removable protective cover, with an inner cavity defined by said inner surface of said backplate, said flat cavity surface said U-shaped platform and said removable protective cover;
said plurality of hemisphere dampers are sorbothane to minimize human error during the diagnostic testing, provided along said U-shaped inner surface of said U-shaped platform base;
a plurality of fixed support pegs supporting said microprocessor, said at least one IMU and said Bluetooth module within said inner cavity an upon said upper surface of said extension panel, providing additional measuring of any and all the angles and deviations along an X, Y and Z axis to the nearest 100^{th} of a degree as said device is runs along a predetermined pathway;
said microprocessor further defining an open-source prototyping platform based upon easy-to-use hardware and software, which enable circuitry to read inputs and turn it into an output, programmable by a user by sending a set of instructions to the microprocessor using compatible language and programming, said microprocessor comprising the main processing means of communicating of all data from said at least one IMU to said Bluetooth module, said Bluetooth module used to relay information and data from the said at least one IMU to said microprocessor and to said external computer or other visual graphic display, with said Bluetooth programmed with compliant and appropriate software convert information and data into a visual chart, graph or other visual readout to determine if an abnormal findings are obtained during operation of said device, wherein said L-shaped platform is used for diagnostic testing of fractures or dents in outer surfaces in small diameter beams and pipes, deflections in outer surfaces of pipes and beams, structural cracks in pipes and beams, angular irregularities small dimensional objects which are capable of being fully or partially fit within the U-shaped inner surface, cylindrical objects to diagnose uniform outer diameters and surfaces, to determine proper fitting and angle of joints, dimensional lumber straightness and uniformity, and bulging or pressure deviation is pipes, tubing and ducts.

6. A deviation detection scan device providing for the measurement of deviations in linear and planar surfaces of flat or shaped objects, resulting in a visual diagnosis of micro-measured irregularities in said surfaces of said objects, comprising:
a shaped platform defining at least one surface extending a plurality of hemispheric dampeners attached to said surface of said platform;
at least one Inertial Measurement Unit (IMU) supplied as a gyroscope;
a programmable microprocessor;
a Bluetooth module to convey data from said IMU to said microprocessor and to an external visual graphic display device; and
said IMU, said microprocessor and said Bluetooth module attaching as shown in a circuit diagram shown in FIG. 5.

7. The deviation detection scan device as disclosed in claim 6, wherein said microprocessor is an Arduino Uno, which is an open-source prototyping platform based upon easy-to-use hardware and software, which enable circuitry to read inputs and turn it into an output and are programmable by a user by sending a set of instructions to said microprocessor using Arduino language and programming.

8. The deviation detection scan device as disclosed in claim 6, further comprising:
said platform defines a flat base member having an inner surface, an outer surface and extending a side projection, a removable protective cover containing an inner cavity and an upper window section;
a rigid clear plastic window providing a plurality of graphic crosshairs, said clear plastic window set within said upper window section for accurate measurements along a designated pathway on a planar object being measured;
at least one first IMU providing an electronic gyroscope set upon said clear plastic window measuring any and all the angles and deviations along an X, Y and Z axis to the nearest 100^{th} of a degree as said device is runs along a predetermined pathway;
said plurality of hemispheres are sorbothane, providing a vibration damper to said device as said device is run along said planar object, minimizing human error resulting from shaking or unsteady hands and are mounted upon said outer surface of said flat base member;
a plurality of fixed support pegs supporting said microprocessor, said second IMU and said Bluetooth module within said inner cavity, said second IMU providing additional measuring of any and all the angles and deviations along an X, Y and Z axis to the nearest 100^{th} of a degree as said device is runs along a predetermined pathway;
said microprocessor further defining an open-source prototyping platform based upon easy-to-use hardware and software, which enable circuitry to read inputs and turn it into an output, programmable by a user by sending a set of instructions to the microprocessor using compatible language and programming, said microprocessor comprising the main processing means of communicating of all data from said first and second IMU to said Bluetooth module, said Bluetooth module used to relay information and data from the said first and second IMU to said microprocessor and to said external computer or other visual graphic display, with said Bluetooth programmed with compliant and appropriate software convert information and data into a visual chart, graph or other visual readout to determine if an abnormal findings are obtained during operation of said device, wherein said device is used to diagnose scoliosis in children and adolescent without exposure to harmful radiation used in other forms of diagnosis including disruptive diagnostic testing including X-ray, MRI and CT scans and for non-destructive testing of manufactured products to detect fractures or dents in surfaces, including aircraft and automotive surfaces, deflections in pipes and beams, cracks in bridge supports, walls and panels, and other surface irregularities.

9. The deviation detection scan device as disclosed in claim 6, used for detection of irregularities upon inside and outside corners of said object, said device further comprising:
said platform is L-shaped defining a rear panel having an inner surface and an outer surface, an integrated perpendicular extension panel extending from said inner surface, said extension panel defining an upper surface and a lower surface, and a removable protective cover defining an outer surface, said upper surface and said removable protective cover defining an inner cavity;
said plurality of hemisphere dampers are sorbothane to minimize human error during the diagnostic testing provided upon said outer surface of said rear panel, a portion of said inner surface of said rear panel outside said inner cavity, said lower surface of said extension panel and an outer surface of said protective cover;
a plurality of fixed support pegs supporting said microprocessor, said at least one IMU and said Bluetooth module within said inner cavity an upon said upper surface of said extension panel, providing additional measuring of any and all the angles and deviations along an X, Y and Z axis to the nearest 100^{th} of a degree as said device is runs along a predetermined pathway;
said microprocessor further defining an open-source prototyping platform based upon easy-to-use hardware and software, which enable circuitry to read inputs and turn it into an output, programmable by a user by sending a set of instructions to the microprocessor using compatible language and programming, said microprocessor comprising the main processing means of communicating of all data from said at least one IMU to said Bluetooth module, said Bluetooth module used to relay information and data from the said at least one IMU to said microprocessor and to said external computer or other visual graphic display, with said Bluetooth programmed with compliant and appropriate software convert information and data into a visual chart, graph or other visual readout to determine if an abnormal findings are obtained during operation of said device, wherein said L-shaped platform is used for diagnostic testing of a linear inside corner utilizing the sorbothane hemisphere dampers located on said outer surface of said protective cover and said outer surface of said rear panel and, when diagnostic testing of a linear outside corner, said hemisphere dampers on said lower surface of said extension panel and said inner surface of said rear panel are utilized, said device used to determine the integrity and linear formation of each said linear inside and outside corners, said L-shaped platform useful in diagnostic testing of wall formations, structural component alignment, and whether a corner is joined in a straight line or used on cylindrical objects to diagnose uniform outer diameters and surfaces.

10. The deviation detection scan device as disclosed in claim 6, used for detection and diagnosis of irregularities in small diameter pipes and beams said shaped objects, said device further comprising:
said platform is U-shaped and defines a U-shaped platform base with a U-shaped inner surface and a flat cavity surface, a backplate extending from said flat cavity surface, said backplate defining an inner surface and an outer surface, and a removable protective cover, with an inner cavity defined by said inner surface of said backplate, said flat cavity surface said U-shaped platform and said removable protective cover,
said plurality of hemisphere dampers are sorbothane to minimize human error during the diagnostic testing, provided along said U-shaped inner surface of said U-shaped platform base;
a plurality of fixed support pegs supporting said microprocessor, said at least one IMU and said Bluetooth module within said inner cavity an upon said upper surface of said extension panel, providing additional measuring of any and all the angles and deviations along an X, Y and Z axis to the nearest 100^{th} of a degree as said device is runs along a predetermined pathway;
said microprocessor further defining an open-source prototyping platform based upon easy-to-use hardware and software, which enable circuitry to read inputs and turn it into an output, programmable by a user by sending a set of instructions to the microprocessor using compatible language and programming, said microprocessor comprising the main processing means of communicating of all data from said at least one IMU to said Bluetooth module, said Bluetooth module used to relay information and data from the said at least one IMU to said microprocessor and to said external computer or other visual graphic display, with said Bluetooth programmed with compliant and appropriate software convert information and data into a visual chart, graph or other visual readout to determine if an abnormal findings are obtained during operation of said device, wherein said L-shaped platform is used for diagnostic testing of fractures or dents in outer surfaces in small diameter beams and pipes, deflections in outer surfaces of pipes and beams, structural cracks in pipes and beams, angular irregularities small dimensional objects which are capable of being fully or partially fit within the U-shaped inner surface, cylindrical objects to diagnose uniform outer diameters and surfaces, to determine proper fitting and angle of joints, dimensional lumber straightness and uniformity, and bulging or pressure deviation is pipes, tubing and ducts.

11. The deviation detection scan device as disclosed in claim 1, further comprising any other element or combination of elements not previously claimed and as contained within the specification and drawings.
